(19) United States
(12) Patent Application Publication
(10) Pub. No.: US 2004/0229854 A1
(43) Pub. Date: Nov. 18, 2004

(54) Immediate release dosage form for HRT

(76) Inventor: Pieter Haan De, Amsteleindstraat (NL)

Correspondence Address: WILLIAM M. BLACKSTONE, AKZO NOBEL, 1300 PICCARD DRIVE #206, ROCKVILLE, MD, 20850-4373, US

(21) Appl. No.: 10/781,493

(22) Filed: Feb. 18, 2004

Related U.S. Application Data
(62) Division of application No. 09/723,412, filed on Nov. 28, 2000, now abandoned.

(51) Int. Cl. A61K 31/57

(52) U.S. Cl. 514/178

(57) ABSTRACT

It has been recognized that tibolone, which is an agent in the treatment of menopausal complaints and osteoporosis, if formulated with a particle size in a defined range, displays an unexpectedly high bioavailability of one of its estrogenic metabilites, Org A.

### FIELD OF THE INVENTION

The invention pertains to a pharmaceutical formulation comprising, as the active substance, (7α, 17α)-17-hydroxy-7-methyl-19-nor-17-pregn-5(10)-en-20-yn-3-one (tibolone), wherein the formulation is a solid, peroral dosage form of the immediate-release type.

### BACKGROUND OF THE INVENTION

Tibolone is known as a tissue-specific and effective agent that can be used in hormone replacement therapy (HRT) in (post)menopausal women, for the treatment of menopausal and postmenopausal disorders, including climacteric complaints, vasomotor symptoms, osteoporosis, and vaginal atrophy. See, int. al., U.S. Pat. No. 5,037,817 and WO 98/47517

Throughout the past years, it has become known that tibolone displays a relatively complex metabolic pattern. Several compounds different from tibolone itself play a role as active metabolites. This makes it difficult to predict whether two different tibolone products are bioequivalent with each other. Even if it is known practice in the field that bioequivalence in the case of a compound which exerts its activity through a metabolite is to be measured, in vivo, by analysing plasma levels of the metabolite, this is a much more complex matter in the case of a compound as tibolone, which has more than one metabolite.

In order to set a standard for analysing the relative bioavailability of a drug substance upon oral administration, a customary method is to choose a model in which, for a dosage amount well within the therapeutic window, a maximum exposure to the drug is given. One generally viable way to achieve such maximum exposure is to provide the drug in the form of a solution. The normal expectation is that the bioavailability of the drug in solid form will be lower than that in the case of the solution. See, int. al., Robert E. Notari, Biopharmaceutics and Clinical Pharmacokinetics, 4^{th} edition (1987) p140.

In the case of tibolone, this experiment led to an unexpected result, on the basis of which the present invention was made.

For different solid formulations, an analysis was made of two of the main metabolite compounds, viz. 3α-OH tibolone (hereinafter referred to as “Org A”), and 3β-OH tibolone (hereinafter referred to as “Org B”). The finding is that one of these, Org A, turned out to become better available as compared to the solution, dependent on the particle size of tibolone. As this increase of bioavailability does not hold for the other above-mentioned metabolite, Org B, obvious explanations could not be given.

Besides being an unexpected result per se, this is an improvement of the solid pharmaceutical product as compared to the solution, since Org A is responsible for an important part of the estrogenic effect of tibolone (which, naturally, plays a role in its significant utility as an HRT agent for menopausal patients).

### SUMMARY OF THE INVENTION

The invention thus resides in drug products of the immediate-release type, of the active substance tibolone which show an improvement of Org A availability as compared to a reference situation with maximum exposure, viz. a solution. These drug products are characterized by comprising solid tibolone in a mean particle-size below 22.8 μm as measured by the technique described below. Preferably, the mean particle size is below 20 μm.

Incidentally, with the benefit of hindsight, one can asses that the product as produced in Example 1 of U.S. Pat. No. 5,037,817 satisfies these same criteria. This, however, is to be regarded as truly accidental, since a teaching on the bioavailability of Org A, let alone of the influence of particle size thereupon, cannot be derived from U.S. Pat. No. 5,037,817.

In addition to the above, it was realized that any assessment of bioequivalence of two tibolone products will fail if not Org A is measured. This leads to another aspect of the present invention, which is a method of examining whether one tibolone product is bio-equivalent with another tibolone product, and which resides therein that bio-equivalency is determined in vivo by analysing plasma levels of one or more metabolites of tibolone, one of which being Org A.

### DETAILED DESCRIPTION OF THE INVENTION

The main requirements for the present invention include that a solid pharmaceutical composition of tibolone is provided, and that tibolone, when contained in the dosage form, has a particle size as defined above.

Tibolone being a known substance, it can be prepared in a known manner. In this respect reference is made to U.S. Pat. Nos. 3,340,279, 4,701,450, and 5,037,817.

Once the person skilled in the art appreciates that tibolone is to be provided in a certain particle size, he or she knows the general measures that can be taken to achieve this. A simple, though laborious and not economical method is to just produce tibolone in any solid form and screen the obtained particles using adequate mesh sizes so as to shift out the particles that are too large. An alternative method is to first dilute the active substance with a pharmaceutically acceptable carrier (e.g. a base granulate normally used for tabletting or filling of capsules), and then screen the product. In that case the carrier materials effectively work as a processing aid in reducing the size of the active substance (the final maximum particle size of the active substance remains to be determined by the mesh-size of the screen). The necessary equipment as well as these screening methods are known to the skilled man. Another method, also known in general to the skilled person, is to again just produce tibolone in any solid form, and then subject it to a milling step wherein the required particle size is obtained. Suitable milling equipment is known to the skilled person. Instead of first producing tibolone and then rendering it into the right particle size, one can also take measures during synthesis and, particularly, crystallisation, to actually obtain tibolone in the desired particle size. The exact conditions required for this, will depend on the equipment and reaction circumstances used. As a general textbook reference on providing particles of a desired size, it is referred to Gennaro et al., Remington's Pharmaceutical Sciences, (18th ed., Mack Publishing Company, 1990, pages 1436-1437 and 1615-1632.

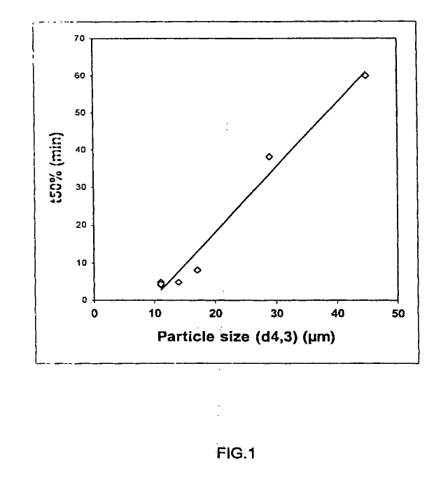

In order to avoid undue experimentation, one can conduct a relatively simple test as to whether the particle size of tibolone, once incorporated into the solid, peroral, immediate-release dosage form (be it a compressed tablet, a capsule, or else), is adequate. This is a dissolution test, standardized to a dosage of 2,5 mg tibolone. The dosage form to be tested is put into a dissolution medium (aqueous sodium lauryl sulphate solution 0.25%). By means of a suitable technique, such as HPLC, it is measured at fixed points in time how much tibolone has been dissolved in the medium. This enables determining the dissolution rate of tibolone, given by the t₅₀ value, i.e. the point in time at which 50% of the tibolone, i.e. 1.25 mg, has been dissolved. A t₅₀ value of below 23.1 minutes correlates with a particle size of tibolone in compliance with the invention. It is imperative that the test be standardized to a dosage of 2.5 mg. This means that in the case of, e.g., a 5 mg tibolone tablet, one has to break it into two parts before conducting the test. Or, e.g., in the case of 1.25 mg or 0.625 mg tibolone dosage units, one has to take two, respectively 4, of such dosage units in the (also standardized) dissolution medium. FIG. 1 provides the correlation between tibolone particle size and the dissolution-rate (t_{50%}) of a solid, immediate-release peroral dosage unit (a tablet) comprising tibolone. Such a correlation is generally valid in the case of immediate-release dosage forms. As indicated above, t_{50%} is the time needed for 50% of the drug to dissolve in vitro. The t_{50%} was calculated by means of linear interpolation between the first point >50% of the drug dissolved, and the previous point, sampling points being 5, 10, 15, 30, and 45 minutes after start.

If the tibolone has been provided in the adequate particle size before mixing with excipients, it can thereafter be mixed with excipients. Eventually, the mixture of tibolone and excipients (how ever obtained) can be incorporated into solid pharmaceutical dosage units in known manner. The dosage amount of tibolone therein will usually be of the order of 0.3 to 5.0 mg per dosage, more particularly 0.625, 1.25 or 2.5 mg.

The pharmaceutical dosage units of the present invention will generally take the form of tablets or capsules, but other solid or dry pharmaceutical preparations are included.

Methods for making such dosage units are well known. For example in the above-mentioned standard English language text Gennaro et al., Remington's Pharmaceutical Sciences, reference is made especially Part 8: Pharmaceutical Preparations and Their Manufacture), methods of making tablets, capsules and pills and their respective components are described.

Three methods of making tablets and capsules include the wet-granulation, dry-granulation, and direct compression methods.

Wet-granulation methods involve weighing out ingredients (actives and excipients, including a solvent), mixing the ingredients, granulating them, screening them damp, drying them, dry screening, lubrication, and compressing the resultant admixture into tablets or filling capsules with it. Such procedures result in tablets or capsules having at least adequate homogeneity and stability.

Direct compression methods involve weighing out direct-compression vehicles (including carriers) and active ingredients, mixing of the ingredients, lubrication, and compressing the resulting admixture into tablets, or filling capsules with it.

Carriers for active substances in pharmaceutical dosage units are generally known to the skilled man, and do riot require separate elucidation here.

Wet granulation distinguishes from dry granulation and dry-mixing in that a liquid (such as water, preferably containing a binder) is applied in wet granulation to produce agglomeration or granules.

The most widely used granulation methods in the pharmaceutical industry are the fluidized bed granulation and the Net-massing method in which a liquid is added to a powder or granulate in a vessel equipped with any type of agitation that will provide granules or agglomerates. Various operations can be recognised in the wet (massing) granulation, including milling of excipients, mixing of milled powders, preparation of binder solution, mixing the binder solution with the powder mixture to form the wet mass, granulation of the mass, coarse screening of wet mass, drying moist granules, and screening dry granules. It is obvious that, depending on the selected excipients and the size of the batch and the selected equipment, some of the operations can be combined or are not required or particular operations can be included. General methods of preparing granules are for instance described in Pharmaceutical Dosage Forms: Tablets (Volume I). Ed. H. A Lieberman, L. Lachman, J. B. Schwartz (1989), Marcel Dekker Inc. New York and Basel pp. 131-190.

Advantages of wet granulation include improvement of the cohesiveness and compressibility of powders, a good particle size distribution, reduction of a great deal of dust and airborne contamination, prevention of segregation of components.

Small-scale production can be achieved by mixing and wetting the mass in mortars or stainless steel bowls, whereas for larger quantities twin-shell blenders, double-cone blenders, planetary mixers, rotary granulators, high shear mixers and fluid-bed granulation equipment can be applied. General mixing methods are disclosed in Pharmaceutical Dosage Forms (Volume 2). Ed. H. A. Lieberman, L. Lachman, J. B. Schwartz (1990), Marcel Dekker Inc. New York and Basel pp. 1-71. The dry excipients and, optionally, active ingredients are mixed in a suitable mixer, preferably a mixer in which both mixing and granulating can be performed, for instance a Gral high shear mixer, after which an aqueous binder solution is added. Another preferred method is suspending the active ingredients into the aqueous binder solution, which suspension is added to the dry mixture of excipients and granulated.

Granulates, tablets, and capsules prepared by wet-granulation or direct compression consist of several inert materials that can be found in conventional solid oral dosage forms in general. The ingredients can be classified in excipients which help to impart satisfactory processing and compression characteristics to the formulation like diluents, stabilising agents, binders, glidants and lubricants and in excipients to give the desirable physical characteristics to the finished tablet like disintegrants and colours. If required the tablets can be provided with a film coat, for instance as disclosed in Pharmaceutical Dosage Forms (Volume 3). Ed. H. A. Lieberman, L. Lachman, J. B. Schwartz (1990), Marcel Dekker Inc. New York and Basel pp. 93-125.

Diluents (“filling excipients”) usually make up the major portion of the carrier. Direct compression carriers are described in the same textbook, Volume 1, second edition, Chapter 4, pages 195-246. The direct compression carriers can be classified into groups including water soluble polyalcohols such as lactose (including spray-dried lactose and anhydrous lactose), and polysaccharides such as the group of celluloses (e.g. Avicel® PH 101, PH 102, and PH 200, purified wood cellulose), and the group of water-insoluble starch products according to the invention (e.g. Starch 1500, potato starch, corn starch, wheat starch, including modified starches, agglomerated starches, granulated starches).

Binding agents or adhesives are used as substances that bind powders together and provide cohesiveness to the granulates and tablet formulation. Binders can be added dry and blended with the diluents and, optionally, the drug. In this case binders are activated by addition of water or other solvents. In other manufacturing procedures, the adhesives are dissolved or slurried in a liquid and, in this form, added to the mixed powders. Conventional binders include gelatine, water soluble modified starch, and sugars as sucrose, glucose, dextrose, molasses and lactose. Natural and synthetic gums which have been used include tragacanth, magnesium aluminium silicate, acacia, ammonium calcium alginate, sodium alginate, carboxymethylcellulose, hydroxypropylcellulose, methylcellulose, hydroxypropylmethyl-cellulose, polyvinylpyrrolidone, polyethylene glycol and clays like Veegum.

Depending on for example the solubility of the binders in the various liquids, the binder can be added to the powder mix as a solution in water, or a water-solvent mixture.

In addition to the stabilising effect of the present invention, stabilising agents can be added to further reduce decomposition of tibolone if desired. Examples of such stabilising agents are of the group of antioxidants (such as ascorbyl palmitate and ascorbyl stearate) and the group of water soluble chelating agents (such as sodium EDTA and sodium ascorbate).

Materials to improve the flow characteristics are referred to as glidants. As an example, silicon dioxide, magnesium lauryl sulfate, magnesium aluminium silicate, magnesium oxide, talc or clays can be incorporated into the formulation to reduce interparticulate friction and to eliminate the problems associated with the flow of materials from larger to smaller apertures in the tablet presses.

Disintegrants (commonly used in immediate-release dosage forms, and known to the skilled person) can also be used in the dosage forms of the invention.

Before filling capsules or sachets, or compressing tablets, lubricants are mostly added to prevent friction and wear during processing. Some of the lubricants also demonstrate anti-adherent properties that can be relevant in case of sticking of tablet granulations to the faces of the punches and the die walls. Examples of the group of lubricants are the metallic stearates (magnesium stearate), talcum, stearic acid, sodium stearyl fumarate, hydrogenated vegetable oil, and high melting point waxes.

As indicated above, the invention also pertains to a method of examining whether one tibolone product is bio-equivalent with another tibolone product. Described more fully, this is a method of determining by means of an in vivo bioequivalence study whether a drug product comprising solid tibolone is bioequivalent with another drug product comprising tibolone which is used as a standard tibolone product, the method comprising the steps of administering to a suitable test panel in a comparative trial the tibolone product of which the bioequivalence is to be determined and the standard tibolone product conducting an in vivo bioequivalence study, and measuring plasma levels of a measurable active substance, characterized in that the active substance is Org A. This method finds utility in the marketing approval of copies of existing market products comprising tibolone. In this respect, one can also view this as a “method of doing business.” It is known in the pharmaceutical arts to try and obtain regulatory approval for a drug product through the filing of an ANDA (Abbreviated New Drug Application), referring to an original (standard) drug product on the basis of the same active substance. Thereby it is shown try means of a comparative trial that the ANDA drug product is bio-equivalent with the standard drug product by analysing and comparing plasma levels of an active compound present in the blood. It is known that, if the compound itself is not suitable for analysis (either because it cannot be determined per se, or because it is metabolized), another compound should be round for analysis (e.g. the active metablite). The problem with doing this for tibolone, is that the metabolic pattern is too complicated to simply select which compound to analyse. By virtue of the present invention, it has now been found that the relevant analysis is that of Org A.

Assays

From the above, it is clear that several assays are used in describing the present invention. These are, in arbitrary order, the measurement of mean particle-size, the dissolution test referred to above, the assessment of bio-equivalence. These will be described hereinbelow:

Particle Size Measurement

Principle:

The particle size distribution is determined by Laser Diffraction Spectroscopy.

Equipment:

Laser diffraction particle sizer equipped with a sample dispersion unit.

Reagents

1. Polysorbate 80, analytical grade.

2. Water, purified quality (concentration of particles >10 μm is <1 per mL).

3. Polysorbate solution 0.05%.

Dissolve 0.5 gram of polysorbate 80 (1) in 1.0 liter of water (2).

4. Suspension liquid.

Add 50 mg of tibolone sample to 500 mL of polysorbate solution (3) and saturate by stirring for at least 60 minutes.

Filtrate the supernatant liquid and use the filtered solution.

Instrument Conditions:

Particle sizer Malvem Mastersizer S or equivalent

Lens type: 300RF (range 0.05 μm-900 μm)

Active beam length: 2.4 mm

Sample dispersion unit: Malvem MS1 or equivalent

Sample flow rate: ¾ of maximum flow rate

Sweeps

sample number: 10000

background number: 10000

Presentation: Fraunhofer

Analysis model: Polydisperse

Sample Suspension:

Homogenize the sample in the sample container.

Transfer about 30 mg of the sample into a 10 ml glass container.

Add about 2 mL of suspension liquid (4) and close the container.

Suspend the sample in an ultrasonic bath to complete deagglomeration.

Measurement:

Fill the sample dispersion unit with suspension liquid (4) and measure the blank background.

Add the required amount of the homogeneous sample suspension dropwise into the sample dispersion unit, and analyse.

Calculation:

Calculate the mean particle size in which the d_{(4,3)}=volume−mean diameter of the particles in μm.

Dissolution Rate of Tibolone.

Principle:

After dissolution, tibolone is determined by reversed phase liquid chromatography.

Equipment:

1. Liquid chromatograph with temperature controlled column compartment and variable wavelength absorption detector.

2. Dissolution apparatus 2 according to USP-DISSOLUTION <711> (paddle).

Reagents and Reference Standards:

1. Water, HPLC grade USP/ACS suitable for gradient elution analysis or equivalent (e.g. milli-Q).

2. Methanol, analytical reagent.

3. Sodium lauryl sulphate solution 99%, Merck or equivalent.

4. Dissolution medium: Sodium lauryl sulphate solution 0.25%.

Remove dissolved air from water (1) by purging with helium or equivalent before adding sodium lauryl sulphate (3).

Weigh 10 g of sodium lauryl sulphate (3) and dissolve in 2.0 liter of hot water (1).

Cool to room temperature and dilute to 4.0 liter with water (1) at room temperature.

Mix carefully, in order to prevent the formation of lather.

5. Tibolone, reference standard.

6. Suitable sinkers in the case of capsules

Reference Solution (in Duplicate):

Accurately weigh about 25 mg of tibolone (5) and transfer into a 50 mL volumetric flask.

Dissolve in and dilute to volume with methanol (2).

Transfer 1.0 mL of this solution into a 100 mL volumetric flask.

Dilute to volume with dissolution medium (4).

Mix carefully, in order to prevent the formation of lather.

Sample Preparation:

Drug substance:

Accurately weigh about 2.5 mg of tibolone and transfer into a gelatine capsule. Add about 20 glass beads, close the capsule and mix gently.

Bring the capsule into the dissolution medium using a pair of tweezers and open the capsule to start dissolution.

For a pharmaceutical dosage form tested, the final amount of tibolone added to the dissolution medium must be 2.5 mg. If the dosage contains less than 2.5 mg tibolone per dosage unit extra dosage units should be added to the medium.

Tablet:

Add a tablet to the dissolution medium to start dissolution.

Capsule:

Place the capsule in a suitable sinker and add the sinker with capsule to the dissolution medium to start dissolution.

Test Solution:

Prepare the test solution according to USP-Dissolution <711>.

Withdraw about 10 ml of the test solution using a glass syringe equipped with stainless steel cannula (or equivalent glass device).

Immediately centrifuge the solution and use the clear supernatant.

Dissolution Conditions:

Apparatus: USP-Dissolution apparatus 2

Dissolution medium: see reagents no. 4

Volume of Dissolution medium: 500 ml

Temperature: 37 0.5° C.

Speed: 0.83 Hz (50 r.p.m.)

Sampling time: 5, 10, 15, 30, and 45 minutes after start.

Chromatographic Conditions:

Column: Hypersil ODS, 5 μm, 100×4.6 mm or equivalent

Column temperature: 40° C.

Mobile phase: methanol+water (77+23 v/v)

Flow rate: 0.5 ml/min

Detector: variable wavelength absorption detector, 205 nm, abs. range about 0.1 AUFS

Injection volume: 200 μl

Number of injections: reference solutions: 3

test solutions: 2

dissolution medium: 1

System Suitability Test:

Indicating retention time for tibolone is 5.0 minutes.

Alter the water concentration of the mobile phase if any impurity coelutes with tibolone.

Criteria:

1. Relative standard deviation of injections of the reference solutions: RSD 3%.

2. Ratio of the mean response factors of the 2 reference solutions: 0.97 Q 1.03.

3. Response of any peak in the chromatogram of the dissolution medium at the position of tibolone: <3% with respect to the response of the tibolone peak in the chromatogram of the reference solution.

Calculation:

Calculate the amount of tibolone dissolved in the sample.

Bioequivalence

Assay for Org A

For the determination of Org A, deuterated Org A can be used as internal standard. Plasma samples can be extracted with solid-phase extraction, thereafter silylated and quantified with capillary gas chromatography with mass spectrometric detection in the Cl-mode. The lower limit of quantification was 0.1 ng/ml.

In Vivo Study

To assess the bioequivalence between a tibolone product (indicated as test treatment) and a reference treatment (such as Livial™ or Xyvion™) a single-dose (2.5 mg) 2-way crossover study should be performed. The wash-out period between the two treatments should be at least seven days. The total number of subjects should be at least 22, i.e. 1 subjects per sequence. Subjects should be postmenopausal women, aged 55-65, and physically and mentally healthy. Following single-dose administration, blood samples should be taken at 0 (pre-dose), 0.5, 1, 1.5, 2, 3, 4, 6, 8, 12, 16, 20 and 24 hours post-dosing. The Org A concentrations should be determined in plasma according to the method described earlier. Based on the Org A plasma concentrations the peak concentrations (Cₘₐₓ) and the area under the plasma-concentration-versus-time curve from zero to the time point of the last measurable concentration (AUC₀₋ₜₗₐₛₜ) as well as the area under the plasma-concentration-versus-time curve from zero extrapolated to infinity (AUC_{0-∞}) should be calculated. Bioequivalence should be assessed based on Cₘₐₓ, AUC₀₋ₜₗₐₛₜ and AUC_{0-∞} using 90% confidence intervals for the ratio of the test and reference treatment derived from an Analysis of Variance on logₑ-transformed parameter values. The formulations are considered to be bioequivalent if the 90% confidence intervals for the above mentioned ratios of Cₘₐₓ, AUC₀₋ₜₗₐₛₜ and AUC_{0-∞} are fully contained within the acceptance range 0.80-1.25.

The invention will be illustrated hereinafter with reference to the following Examples and the Figures.

### EXAMPLE 1

Solid dosage forms of tibolone are made by a process similar to that described in U.S. Pat. No. 5,037,817.

Composition of the Tablets:

| Ingredient | Quantity | |
|---|---|---|
| Tibolon | 2.5 | mg |
| Potato starch | 10 | mg |
| Ascorbyl palmitate | 0.2 | mg |
| Magnesium stearate | 0.5 | mg |
| Lactose | 86.8 | mg |
| Tablet mass | 100 | mg |

A basic granulate is prepared by granulation of a mixture of lactose (diluent), potato starch (90% of the total amount; disintegrant) and potato starch mucilage (10% of the total amount; binder) in a fluid bed granulator. After granulation, the basic granulate is passed through a sieve. Part of the granulate (approx. 10% w/w of the final batch size) is mixed with tibolone and ascorbyl palmitate using a suitable blender and then passed through a premix sieve. The tibolone premix and the remainder of the basic granulate are mixed in a suitable blender. Magnesium stearate is added and mixed. The mixture is compressed into 100 mg tablets.

Thus four batches have been prepared, the only material difference between them being analysed as the particle size of tibolone. See the table below.

### EXAMPLE 2

A solution of tibolone is made as follows. Tibolone is dissolved into Ethanol 96%. The final concentration of tibolone in solution is 1 mg/ml. The solution is mixed and filled in 5 ml vials; 2.5 ml/vial. Dilute instruction: The content of the vial must be diluted with 2.5 ml of solvent (water for injection) up to a total volume of 5 ml before use.

### Table of Tibolone Drug Product Batches

| | Initial particle size | Premix | Mean particle size of tibolone in |
|---|---|---|---|
| Batch | tibolone D_{(4,3)} (μm) | sieve (μm) | dosage form (μm) |
| A | 14 | 250 | 14,4 μm |
| B | 45 | 125 | 19,3 μm |
| C | 102 | 125 | 22,8 μm |
| D | 102 | 250 | 48.2 μm |

| | |
|---|---|
| S | Solution according to Example 2 |

### EXAMPLE 3

The tibolone batches of Examples 1 and 2 are tested for the relative bioavailability of Org A. This test is conducted in the form of the single-dose (2.5 mg) 2-way cross-over study as described hereinbefore, exactly complying with the requirements thereof. The results are depicted in the Figures.

FIG. 1 provides the correlation between tibolone particles size and tablet-dissolution described hereinbefore.

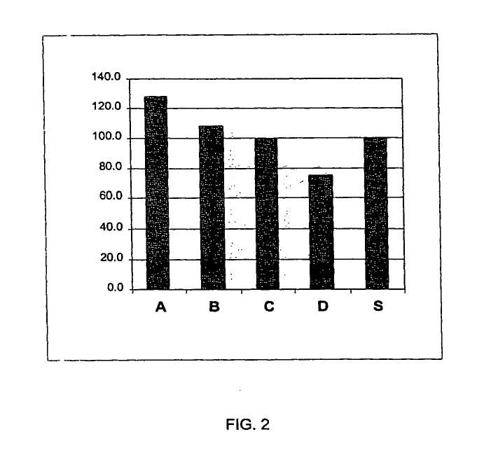

In FIG. 2 the relative bioavailability of Org A is shown. On the Y-axis is depicted the relative Area Under the plasma-concentration versus time curve (AUC), with the solution (batch S) being the reference at 100%. From the picture it is clear that batches A, B and C show an unexpected bioavailability of Org A at least equal to that of the solution batch S.

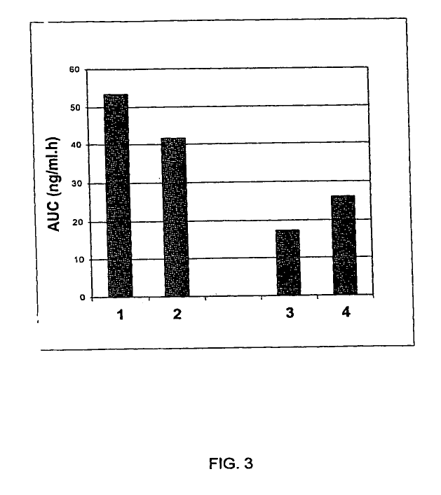

In FIG. 3 the AUC of Org A and Org B is shown for batches A and S, as follows:

(1) is a bar indicating the AUC for Org A after the administration of batch A;

(2) is a bar indicating the AUC for Org A after the administration of batch S;

(3) is a bar indicating the AUC for Org B after the administration of batch A;

(4) is a bar indicating the AUC for Org B after the administration of batch S.

From the relative height of the bars, one can see that the effect of enhanced bioavailability of Org A resulting from the administration of solid tibolone having the appropriate particle size, is not observed for Org B.

1-12. (Cancelled, without prejudice or disclaimer)

13. A method of treatment or prevention of menopausal complaints, comprising administering to a patient in need thereof an effective amount of a pharmaceutical formulation in the form of an immediate-release peroral dosage unit, comprising tibolone, wherein the pharmaceutical formulation has a bioavailability, in vivo, of 3^{α}-OH tibolone at a level above that provided by a solution of tibolone, wherein the amount of tibolone in the immediate-release peroral dosage unit and in the tibolone solution are substantially the same.

14. The method of claim 13, wherein the tibolone in the immediate-release peroral dosage unit has a mean particle size below about 22.8 μm.

15. The method of claim 14, wherein the tibolone in the immediate-release peroral dosage unit has a mean particle size below about 20 μm.

16. The method of claim 13, wherein the rate of dissolution of tibolone from the pharmaceutical formulation is such that when the pharmaceutical formulation is subjected to a dissolution-test, wherein a sample of said pharmaceutical formulation containing 2.5 mg of tibolone is exposed to a 0.25% sodium lauryl sulfate solution and the dissolution rate is measured, a t₅₀% value about 23.1 minutes is obtained.

17. The method of claim 16, wherein the tibolone in the immediate-release peroral dosage unit has a mean particle size below about 22.8 μm.
